(19) United States
(12) Patent Application Publication
(10) Pub. No.: US 2004/0216737 A1
(43) Pub. Date: Nov. 4, 2004

(54) System for anesthetizing laboratory animals

(76) Inventors: Leslie B. Anderson, Easton, PA (US); Alexis Agelan, Philadelphia, PA (US); James Eldon, Barto, PA (US)

Correspondence Address: DANN, DORFMAN, HERRELL & SKILLMAN, 1601 MARKET STREET, SUITE 2400, PHILADELPHIA, PA, 19103-2307, US

(21) Appl. No.: 10/860,230

(22) Filed: Jun. 3, 2004

Related U.S. Application Data
(63) Continuation-in-part of application No. 10/201,848, filed on Jul. 23, 2002, now Pat. No. 6,776,158.
(60) Provisional application No. 60/308,036, filed on Jul. 26, 2001.
(60) Provisional application No. 60/518,588, filed on Nov. 7, 2003.

(51) Int. Cl. A61M 15/00; A61M 16/10

(52) U.S. Cl. 128/203.12

(57) ABSTRACT

A method and apparatus for anesthetizing one or more laboratory animals is provided. An anesthetic component is introduced into a pressurized gas stream for delivery to laboratory animals. In one embodiment of the invention, a solo apparatus is used to administer anesthetic component to a single animal. The apparatus may also include a group apparatus connected to the pressurized gas stream that administers anesthesia to one or more animals simultaneously. One or more solo and/or group apparatuses may be used to administer anesthetic components to different animals at various dosages. The apparatuses of the present invention include a non-rebreathing feature that uses a vacuum-sensitive check valve. The valve permits the delivery of anesthetic gas to the animal only when the animal inhales. The present invention may also include a thermoregulatory system that transfers heat to animals as they receive anesthesia, and an exhaust system to capture exhaust gases.

### RELATED APPLICATIONS

This application is a continuation-in-part application of U.S. application Ser. No. 10/201,848 filed Jul. 23, 2002, which claims the benefit of U.S. Provisional Application No. 60/308,036, filed Jul. 26, 2001. This application further claims the benefit of U.S. Provisional Application No. 60/518,588 filed Nov. 7, 2003. All of the foregoing references are incorporated herein by reference.

### FIELD OF INVENTION

The present invention relates to the administration of anesthesia to laboratory animals, and more particularly relates to methods and apparatuses for safely and efficiently delivering gaseous anesthetic components to laboratory animals prior to and during surgical and testing procedures.

### BACKGROUND OF THE INVENTION

Prior to the present invention, laboratory personnel had to devise their own systems to safely and efficiently anesthetize laboratory animals. Such self-devised systems frequently fail to administer gaseous anesthesia in-a properly controlled fashion and therefore can lead to situations in which a laboratory animal receives either an insufficient amount of anesthetic or an overdose of anesthetic. In cases where a laboratory animal receives an insufficient amount of anesthetic, the under-anesthetized animal may suffer unnecessarily during a laboratory procedure, such as during a surgical procedure. In severe cases, the laboratory animal may become conscious during a surgical procedure, thereby jeopardizing its safety and the successful completion of the procedure. In cases where a laboratory animal receives an overdose of anesthetic, the animal may take longer to recover from the procedure and, in the worst case scenario, may die as a result of a lethal overdose. Either of these situations would adversely influence the successful completion of the procedure.

Many of the problems encountered in prior art anesthesia systems are attributed to the anesthesia masks that deliver gas to the animal's breathing passage. Most prior art anesthesia masks fail to control the flow of anesthetic gas to the animal. In many cases, the mask supplies a constant flow of anesthetic gas across the animal's nose and mouth. Since the flow of anesthetic gas is not synchronized with the animal's breathing, the animal is bombarded with pressurized gas when the animal attempts to exhale. This can impede or even prevent the animal from exhaling anesthetic gas and carbon dioxide from its lungs. Prior art enclosures also feature a large volume of dead space between the source of anesthetic gas and the animal's breathing passage. The dead space usually functions as a two-way conduit that introduces anesthetic gas to the animal, and receives carbon dioxide exhaled by the animal. If the volume of dead space is too large, the carbon dioxide may not be fully displaced and removed from the enclosure when the animal exhales, allowing the animal to inhale or “rebreathe” gases in the enclosure. Over time, the rebreathing of anesthetic gas and carbon dioxide can harm or kill the animal.

Prior art anesthesia masks are typically not designed to restrict movement of the animal's head, and many devices allow the animal to move while being anesthetized. This movement can disrupt the administration of anesthetic gas and interfere with the test or procedure being implemented. Head movement is especially troublesome during stereotaxic procedures, where the animal's head must be immobilized.

Prior art systems that deliver a constant flow of anesthetic gas are highly inefficient, since much of the gas flow bypasses the animal when the animal exhales. Gas that bypasses the animal results in costly waste of anesthetic gas mixture, and can create an unsafe environment for individuals working around the anesthesia system. Uncontrolled releases of anesthetic gas can subject personnel to levels of isoflurane or other components that exceed safety exposure limits. To address this problem, laboratory personnel have used laboratory fume hoods to remove fugitive gas emissions and limit inhalation of anesthetic gas. Since laboratory fume hoods are generally not designed for working with laboratory animals, particularly when a procedure involves the simultaneous manipulation of several animals, conventional fume hoods are cumbersome and ineffective in preventing the release of unsafe levels of anesthetic gas into the laboratory environment. Smaller fume hoods can make it difficult or impossible to arrange equipment, such as manifolds and tubing, discouraging the use of fume hoods.

Prior art anesthesia systems also fail to maintain the body temperature of the animal prior to and during testing or surgery. Prior to the present invention, laboratory personnel had to devise their own systems for maintaining the normal body temperature of a laboratory animal before, during, and after an experimental procedure. If an experimental procedure requires surgery, then maintenance of a laboratory animal's normal body temperature is essential for the animal's survival. Loss of thermoregulatory homeostasis can lead to physiological shock, which is frequently fatal.

Based on the foregoing, prior art anesthesia systems and breathing devices fall short of the needs of technicians desiring a system for administering anesthesia in a safe and controlled manner, while maintaining the position of the animal's head.

### SUMMARY OF THE INVENTION

The present invention provides a method and apparatus for safely and efficiently delivering anesthetic gas to laboratory animals prior to and during a surgical or testing procedure. Moreover, the invention enables laboratory personnel to perform multiple procedures on different animals simultaneously, thereby reducing the time required to accomplish these procedures. Portable components of the apparatus maximize the ease with which the apparatus can be used with other laboratory equipment. The present invention may be comprised of one or more components that may be selectively used to perform a variety of tests and procedures.

A gaseous anesthetic supply system may be provided, comprising a gas supply and a first regulator which provides means to control the flow rate of the gas stream. The gas supply may supply a stream of pure oxygen. The anesthetic supply system may further comprise a vaporizer which introduces anesthetic components into the incoming oxygen stream, and a second regulator which controls the flow rate of gaseous anesthetic/oxygen mix for delivery to a laboratory animal. Where it is desired to facilitate the rapid recovery of laboratory animals following anesthesia, the anesthetic gas stream may be altered or bypassed by an alternate gas to facilitate recovery of the animal. For example, the system may include an emergency bypass feature that substitutes pure oxygen for anesthetic gas mix.

Anesthetic gas or alternate gas streams may be delivered to the laboratory animal in multiple ways under the present invention. In one embodiment of the invention, gas is delivered directly to a laboratory animal using a delivery mask that conveys the gas stream to the nose of the animal. Gas may also be delivered to one or more laboratory animals by placing the animal or animals in an enclosed imperforate container or host cage and introducing the gas stream into the cage. Where delivery masks are used, the invention provides a variety of delivery masks designed to fit different animal species that are used in laboratory procedures. The delivery masks are configured to conform to the anatomy of the laboratory animal so that gas is delivered directly to the animal with minimal fugitive emissions of gas. By minimizing fugitive emissions of gas, the volume of gas consumed or wasted is reduced, and the risk of exposing lab personnel to gas emissions is lowered.

A thermoregulatory system may be provided to achieve and maintain a set temperature on the exterior of the animal supports where animals are positioned during procedures. By controlling the temperature of the animal supports, the body temperature of the laboratory animal may be maintained within a desired range to ensure the well-being of the animal and facilitate the successful completion of the procedure.

The present invention may further include an exhaust system that removes exhaled gases from the vicinity of the animal. The exhaust system may include filters, such as activated carbon filters, which capture exhaled gases and contaminants. The filters may be used in connection with delivery masks, host cages or other gas delivery mechanisms to capture exhausted gases and contaminants. This feature further minimizes inadvertent inhalation of exhaust gases by laboratory personnel. Filters may be used in conjunction with indicators or sensors that show the remaining absorptive capacity of the filters, signaling when the filter should be changed. In addition to or in lieu of filters, the exhaust system may also comprise a horizontal negative pressure recapture apparatus to vent exhaled gases and excess anesthetic, thereby ensuring the safety of laboratory personnel.

The anesthetizing system may be configured for use on a laboratory bench. Alternatively, the anesthetizing system may include portable components that permit the system to be easily transported and set up in different locations. In one portable system, anesthetic gas may be delivered through an air exchange chamber to deliver gas as the animal inhales and removes gas when the animal exhales. Exhaled gas may be directed through a filter before being discharged. The gas may be delivered through a mask mounted adjacent to an animal support base. The support base may be connected to a thermoregulatory system to control the animal's body temperature.

The present invention also features a self-regulating on-demand breathing apparatus that administers anesthetic gas to the nose of an animal only when the animal inhales. The breathing apparatus includes enclosure forming a chamber and comprising an inlet hub extending within said chamber. The inlet hub forms an inlet port through a wall of the enclosure adapted to convey the anesthetic gas through the wall into the chamber. A receptacle extends from the enclosure and forms a fluid path between the animal's nose and the chamber. A check valve connects with the inlet hub and extends within the chamber adjacent to the receptacle. The check valve is operable in response to relative pressure in the chamber between an open position, which permits anesthetic gas to enter the chamber, and a closed position, which substantially prevents anesthetic gas from entering the chamber. The check valve is normally biased in the closed position. When the animal inhales, the check valve moves to the open position in response to vacuum pressure created when the animal inhales to administer the anesthetic gas in response to the animal's breathing.

### BRIEF DESCRIPTION OF THE DRAWINGS

The foregoing summary as well as the following description will be better understood when read in conjunction with the figures in which:

FIG. 1 is a diagrammatic view of a preferred system for anesthetizing animals embodying the present invention;

FIG. 2 is a schematic view and block diagram of a gaseous anesthetic system in accordance with the present invention, with outlets to multiple apparatuses for administering an anesthetic gas;

FIG. 3 is a perspective view of components of a gaseous anesthetic system with an anesthesia support base, an anesthetic host cage, an exhaust system, and a thermoregulatory system;

FIG. 4 is an exploded perspective view of a reversible animal support of the apparatus in FIG. 1.

FIG. 5 is an isolated perspective view of the animal support of FIG. 4 showing a channeled surface on which a laboratory animal may be placed.

FIG. 6 is an isolated perspective view of the animal support of FIG. 4 which is inverted relative to the position in FIG. 5 to illustrate a flat surface on which a laboratory animal may be placed.

FIG. 7 is a cross-sectional view of the animal support taken on the line 7-7 of FIG. 6;

FIG. 8 is an exploded view of an anesthetic host cage and support base;

FIG. 9 is a cross-sectional view of the anesthetic host cage of FIG. 8 in an assembled condition;

FIG. 10 is a perspective view of the front of an exhaust system, showing two embodiments of a perforated grid, manifold conduits and an exhaust manifold;

FIG. 11 is an exploded view of one embodiment of the perforated grid having a removable insert exposing an opening into which an anesthetic host cage may be inserted;

FIG. 12 is an assembled view of the perforated grid of FIG. 11 with an anesthetic host cage inserted through the grid;

FIG. 13 is a cross sectional view of the perforated grid and host cage of FIG. 12, showing flow patterns during operation of the exhaust system;

FIG. 14 is a schematic view of the back of the Exhaust System including a perforated grid, manifold conduit, manifold and attachment thereto;

FIG. 15 is an elevational view of a set of gas delivery masks of the present invention;

FIG. 16 is a perspective view of an alternate gas delivery system which includes a non-rebreathing gas delivery mask and an air exchange chamber;

FIG. 17 is a view of the air exchange chamber of FIG. 16 with parts broken away to illustrate its construction;

FIG. 18 is a block diagram of a preferred arrangement of components and connections with a group apparatus and multiple solo apparatuses;

FIG. 19 is an elevational view of an alternate gas delivery system which includes an intubating tube and an air exchange chamber;

FIG. 20 is a perspective view of an alternate gas delivery system which includes a non-rebreathing gas delivery enclosure for stereotaxic and non-stereotaxic procedures;

FIG. 21 is a perspective view of the enclosure used in the gas delivery system of FIG. 20, showing the nose aperture portion of the enclosure;

FIG. 22 is an exploded perspective view of the enclosure used in the gas delivery system of FIG. 20;

FIG. 23 is a perspective view of the enclosure used in the gas delivery system of FIG. 20, showing a sealed portion on the enclosure;

FIG. 24 is a perspective view of the enclosure used in the gas delivery system of FIG. 20, with a part broken away to illustrate an internal component.

FIG. 25 is a perspective view of a base portion of the enclosure in FIG. 20, shown with an exposed cross-section.

### DESCRIPTION OF THE PREFERRED EMBODIMENT

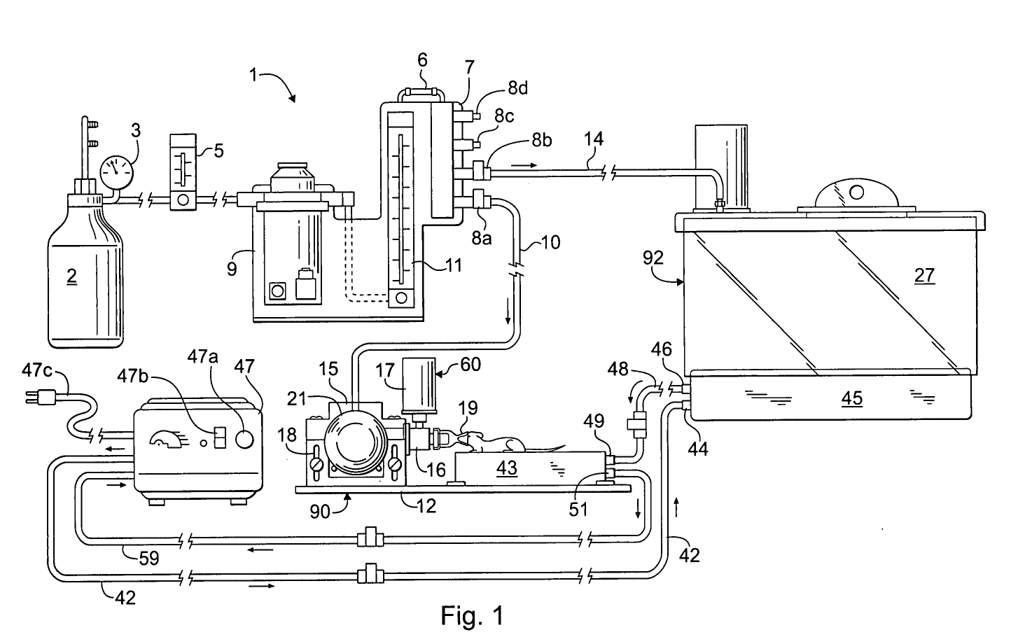

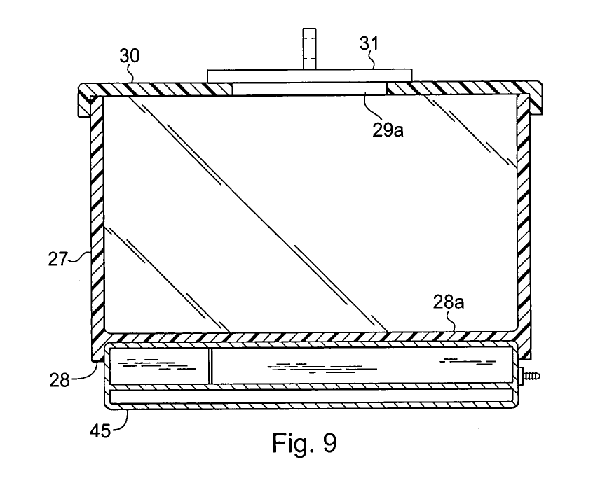

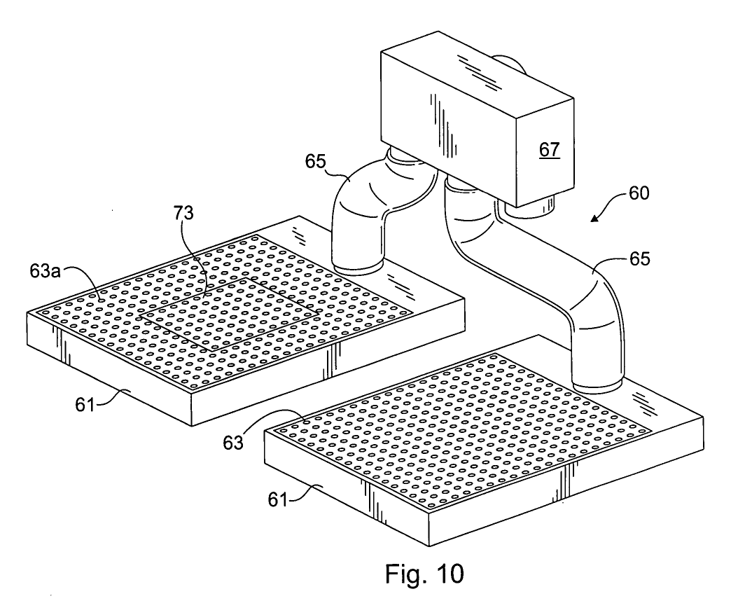

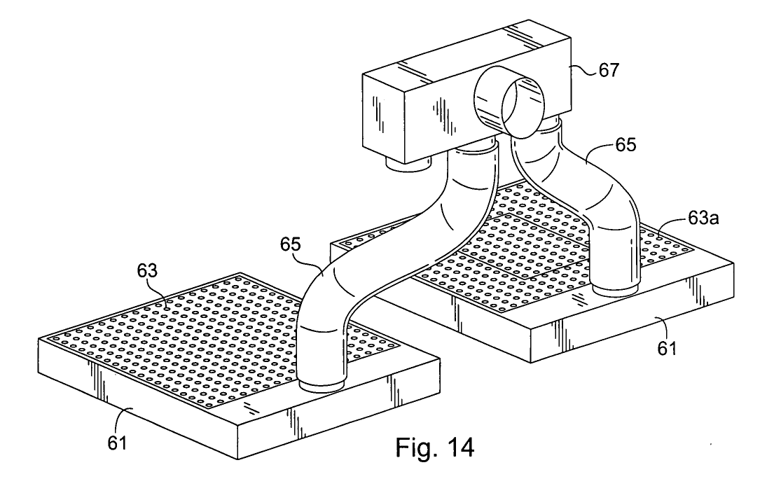

Referring to FIGS. 1-25 generally, and to FIG. 1 in particular, a system in accordance with the present invention is shown and designated generally as 1. The system 1 is particularly adapted for use in laboratories and research facilities in which tests and experiments are performed on animals that require the administration of anesthesia. Such tests and experiments must be performed on a properly anesthetized animal to facilitate successful completion of the procedure and ensure humane treatment of the animal during the procedure. The method and apparatus of the present invention provide means for the controlled delivery of gaseous components for inhalation by laboratory animals, and means for the removal of gases exhaled by the animals. The gaseous components are delivered in a carrier medium, such as pressurized air, oxygen gas, or the like, which is infused with atomized or vaporized anesthetic components. The flow of anesthetic components is controlled to achieve a concentration in the carrier medium that is sufficient to anesthetize a laboratory animal.

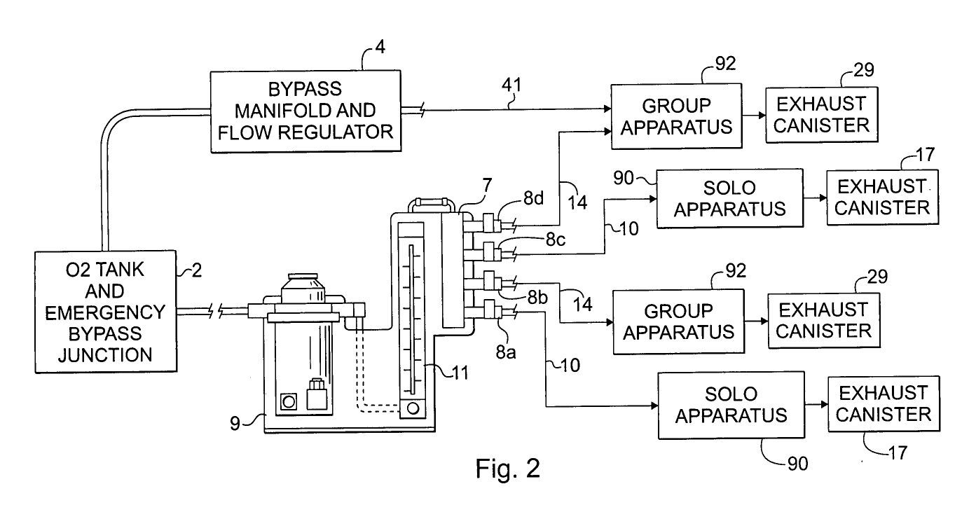

The carrier gas is supplied to the system from a suitable source. Referring to FIGS. 1-2, the carrier gas is supplied from a pressurized gas tank 2 containing oxygen gas. The oxygen gas may be mixed with the anesthetic component in a life support system and supplied through a delivery system. Tank 2 is equipped with a oxygen pressure regulator 3 (for example, Euthanex Model #EZ-220 or EZ-230) to provide means to regulate the flow rate of oxygen leaving the tank and entering the system 1. The flow of the oxygen as it enters the system 1 is detected by a flow meter 5. The oxygen is delivered from flow meter 5 to a vaporizer 9, which atomizes components to facilitate their introduction into the incoming carrier stream. In accordance with the present invention, the system 1 may introduce one of a group of anesthetic components, including but not limited to, isoflurane, halothane, enflurane, and sevoflurane. Vaporizer 9 blends oxygen and anesthetic to a proper ratio to form an anesthetic gas mixture. The vaporizer 9 has a large locking dial with gradations that may be adjusted to control the composition of the gaseous mixture. The anesthetic gas mix is delivered to a gas manifold 7, which has a single inlet port for entry of anesthetic gases and a plurality of outlet ports for supplying the anesthetic gases to the anesthetic delivery system. The manifold 7 may include an optional flow meter 11 to detect and regulate the flow rate of the anesthetic gas mix which exits the vaporizer 9. In FIG. 1, the manifold 7 is shown with four outlet ports 8a-8d. Outlet port 8a is connected to a first apparatus 90, and outlet port 8b is connected to a second apparatus 92, as will be explained in more detail below.

The system of the present invention facilitates the simultaneous performance of a variety of different laboratory procedures. The components of the present invention allow for the administration of anesthetic to a single laboratory animal, or to a plurality of laboratory animals at one time. Referring to FIG. 2, the gas manifold 7 is shown connected to four apparatuses. The system 1 includes two solo apparatuses 90 and two group apparatuses 92. The term “solo apparatus”, as used herein, refers to an apparatus that administers anesthetic to up to one animal, and the term “group apparatus” refers to an apparatus that administers anesthetic to one or more animals simultaneously. The apparatuses 90, 92 each discharge to an exhaust mechanism that captures anesthetic gas exhaled from the animals and any contaminants in the released gas. In FIG. 2, the solo apparatuses 90 are shown discharging to exhaust canisters 17, and the group apparatuses 92 are shown discharging to exhaust canisters 29.

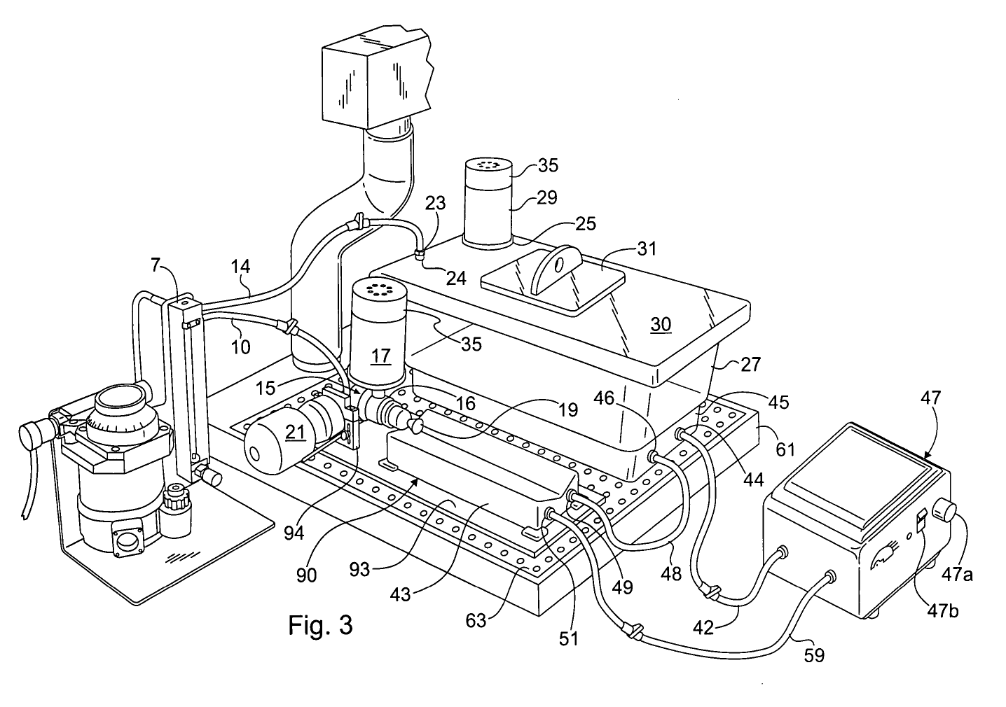

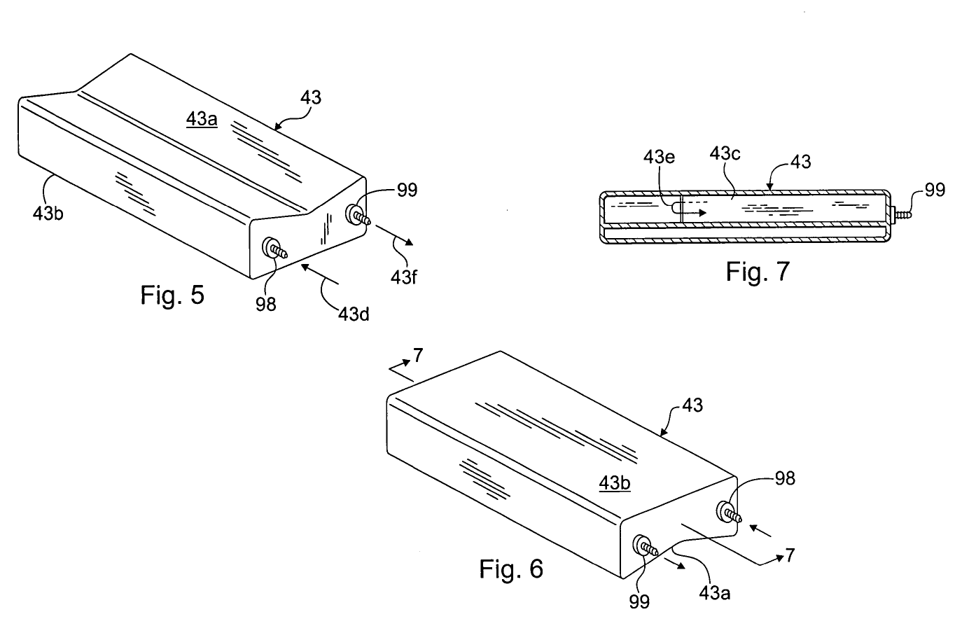

Solo apparatuses may be arranged in a variety of configurations depending on the type of procedure being performed, and other criteria. Referring now to FIGS. 3-7, solo apparatus 90 comprises an animal support base 43 which is configured to support a single laboratory animal during administration of an anesthetic. The base 43 may be formed with different dimensions and geometries depending on the particular species of animal used and the type of procedure being performed. In FIGS. 5-7, the base 43 is shown as a reversible structure having a channeled or depressed surface 43a on one side and a substantially uniform or flat surface 43b on the opposite side. The channeled surface 43a is configured to support a laboratory animal during non-surgical procedures and tests, and the flat surface 43b is configured to support an animal during surgical procedures and tests. In practice, the animal may be placed on the flat side 43b during performance of a surgical procedure or test. When the surgical procedure or test is completed, the animal may be lifted off of the base, and the base 43 may be reversed to expose the channeled side 43a. The animal may then be placed on the channeled side 43a, which will cradle the animal during recovery from anesthesia. The support base 43 may further include mechanisms for restraining the laboratory animal, such as straps or bars.

Referring again to FIG. 1, the outlet port 8a on the gas manifold 7 is connected to a conduit 10 through which anesthetic gas mix is supplied to the solo apparatus 90. Solo apparatus 90 comprises a breather enclosure 15 that delivers an anesthetic gas mix to a laboratory animal. The breather enclosure 15 has an internal air exchange chamber 16 which receives anesthetic gas mix from the conduit 10. Anesthetic gas mix enters the chamber in a controlled manner that prevents harm to the animal and inadvertent release of excess anesthetic gas. In the preferred embodiment, the anesthetic gas mix enters the air exchange chamber through an inlet port and a check valve. The check valve, which will be described in more detail, facilitates controlled inflow of anesthetic gas mix into the air exchange chamber upon inhalation by the animal.

The mask 19 and breather enclosure 15 may be formed of a variety of materials, depending on the application. For example, the mask 19 and breather enclosure 15 may be formed of stainless steel. Alternatively, the mask 19 and breather enclosure 15 may be formed of radiolucent materials to allow use in horizontal or vertical magnetic resonance imaging (MRI) devices. A variety of radiolucent materials may be used, including but not limited to composites and thermoplastics formed of a radiolucent material.

The breathing device may include optional accessories for different procedures. For example, the breather enclosure 15 may have automatic controls which can be programmed to deliver a specified flow rate of anesthetic gas mix appropriate for a specific laboratory animal. Breather enclosure 15 may also include a ventilator to assist the breathing process of a laboratory animal under conditions of deep anesthesia wherein the animal can no longer breathe without assistance. Breather enclosure 15 may further comprise a small soft chamber or bulb 21 which may be used to manually ventilate the respiratory system of a laboratory animal for the purposes of resuscitation.

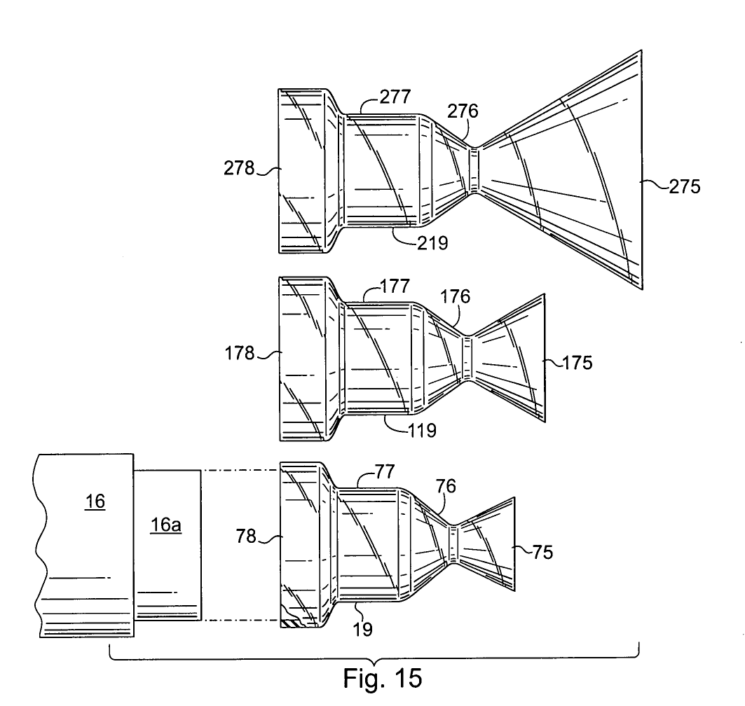

The anesthetic gas mix enters the breather enclosure 15 and flows into an air exchange chamber 16 having a connector 16a. The connector 16a is adapted for coupling with a variety of breathing devices that connect the breathing device with an animal's breathing passage. For example, the connector 16a may connect with a mask, which is used for animals that are breathing on their own. In FIG. 15, the connector 16a is shown with three different cone-shaped or funnel-shaped gas delivery masks 19, 119 and 219. Each of the masks 19, 119, 219 is configured to deliver anesthetic gas mix directly to the nose of a laboratory animal. The masks 19, 119, 219 are each configured and dimensioned to conform to the anatomy of a different animal species. The masks 19, 119 and 219 may be used to deliver anesthetic gas mix to various species, including but not limited to, rabbits, mice, rats, hamsters, guinea pigs, cats, small dogs, reptiles, and birds. The material used to form masks 19, 119 and 219 is preferably transparent so that the animal's breathing can be visually monitored.

For purposes of this description, the component features of mask 19 will be described. The features of mask 119 are identified by reference numbers corresponding to the numbered features on mask 19, with the addition of 100. The features of mask 219 are identified by reference numbers corresponding to the numbered features on mask 19, with the addition of 200. The first end of the mask 19 is comprised of a cone-like enclosure or receptacle 75 configured to fit around the nose of an animal. The anterior end of the animal may be placed adjacent to the mask such that the nose of the animal is disposed within the enclosure 75. The enclosure 75 converges to a small neck which connects to a sheath 76. The sheath 76 is a cone-shaped section having a small diameter end connected with the enclosure 75 and a wider diameter end. The small diameter end of sheath 76 converges toward the enclosure 75 so that gas flow is focused in a relatively narrow stream to the animal's nose. The wider diameter end of the sheath 76 connects to a cylindrical body section 77, which in turn connects to an enlarged diameter fitting 78 at the second end of the mask. The fitting 78 is configured to fluidly connect the mask 19 to the internal chamber of the breather enclosure 15 or other source of gas. The mask 19 may be connected to the breather enclosure 15 with any type of connection. For example, the interior of the second end of the fitting 78 may be slightly larger than the outer diameter of a connector 16a so that the mask is connected to the air exchange chamber 16 by a frictional engagement between the second end of the fitting 78 and the connector 16a. Alternatively, the interior diameter of the second end of the fitting 78 may contain female threading that cooperates and mates with complementary male threads on the exterior of the connector 16a.

The interiors of the fittings 78,178 and 278 are of the same diameter so as to enable substitution of anyone of the masks 19,119 and 219 for another mask, as when the anesthesia system of the present invention is used for different species of laboratory animals. All of the masks are adapted to be mounted on the same connector 16a.

Preferably, an adjustment mechanism is provided to adjust the position of the gas delivery mask 19 relative to the support base 43. In FIG. 1, the breather enclosure 15, air exchange chamber 16 and mask 19 are supported by an adjustment mechanism 18 that is operable to adjust the vertical position of the mask. In this way, the mask 19 may be selectively positioned to accommodate a particular size of animal.

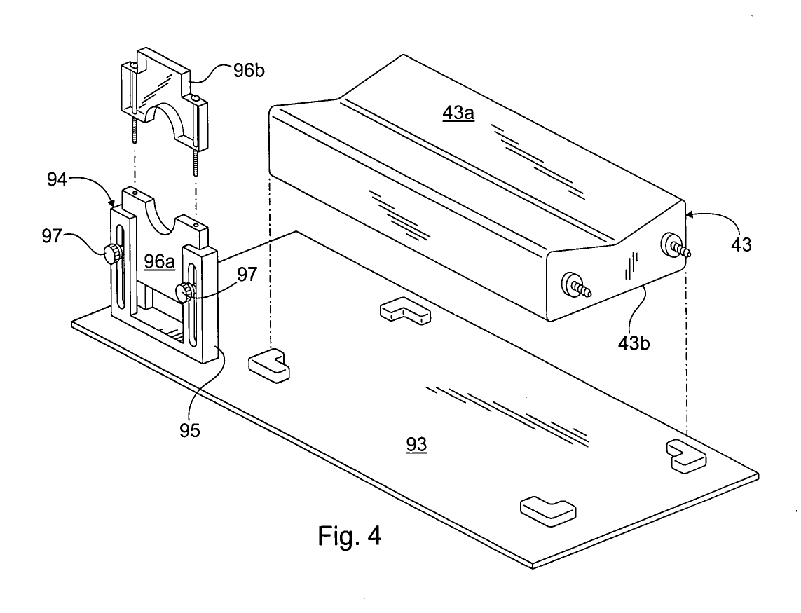

Referring now to FIGS. 3-4, the support base 43 is configured to rest on an animal support 93. The breather enclosure 15 cooperates with a clamp 94 mounted on the animal support 93. The clamp 94 includes an upright frame 95, a first plate 96a and a second plate 96b, as shown in FIG. 4. The plates 96a and 96b connect together, forming an interior aperture that fits around a portion of the breather enclosure 15. As such, the plates 96a and 96b may be connected to form a clamp around the breathing device. The plates 96a and 96b are configured to slide within the frame 95 when clamped around the breather enclosure 15 to adjust the position of the breathing device relative to the support base 43. A pair of thumb screws 97 extend through a pair of slots in the frame 95 and are configured to engage the first plate 96a to control the adjustment of the breathing device relative to the base 43. More specifically, the thumb screws 97 are configured to protrude into and out of engagement with the first plate 96a when the screws are tightened or loosened in the slots of the frame. In this way, the position of breather enclosure 15 relative to the base 43 may be adjusted by loosening the thumb screws 97 in the frame 95. The breather enclosure 15 may be locked into a fixed position by tightening the thumb screws 97 so that the screws engage the first plate 96a and limit further movement of the plates.

One significant feature of the present invention includes a “non-rebreathing” arrangement that prevents harm to the animal. The cone-shaped delivery mask 19 is configured to cover the nose of a laboratory animal, thereby facilitating inhalation of anesthetic gas mix from the air exchange chamber. The cone-shape of the mask 19 conforms with the animal's nose so that the mask leaves very little void space, or “dead space”, between the animal's nose and the inner walls of the mask. This arrangement permits gas to be delivered efficiently to the animal's nose, with minimal accumulation of gas in the mask.

The non-rebreathing gas delivery masks described herein are preferably formed of a flexible material, such as plastic, that can deflect and conform to the contours of an animal's nose. This arrangement provides a good seal to minimize the release of anesthetic gas mix from the mask into the environment.

The air exchange chamber 16 is configured to receive anesthetic gas and carbon dioxide from the mask when the animal exhales. The air exchange chamber 16 includes an exhaust system 60 operable to immediately remove the anesthetic gas and carbon dioxide that accumulates in the chamber, reducing the risk of animal death due to inhalation of excess anesthetic gas and carbon dioxide. Referring to FIGS. 1 and 3, the exhaust system 60 comprises an air filter 17 which is connected to an exhaust port in the chamber of the breather enclosure 15. The filter 17 contains an absorbent, such as charcoal, which provides means to capture anesthetic gas contaminants in the outflow from the breather enclosure 15.

Referring now to FIGS. 1, 8 and 9, the group apparatus will be described in more detail. The group apparatus 92 is adapted to administer an anesthetic component to a plurality of animals. The outlet port 8b of gas manifold 7 is connected to a conduit 14, which provides means to deliver anesthetic gas mix to the group apparatus 92. The group apparatus 92 comprises an anesthetic host cage 27 configured to contain a plurality of laboratory animals at one time. The anesthetic host cage 27 forms an imperforate container having sidewalls 28 and a bottom wall 28a. The sidewalls 28 project downwardly below the bottom wall 28a to form an open bottom pocket 28b. The pocket 28b is configured to fit snugly over a bottom support base 45 to enable heat transfer from the base 45 to the bottom wall 28a. The cage 27 has a cover 30 configured to be placed over the sidewalls 28 to form an enclosure when the sidewalls are positioned on the base support 45. The cover 30 includes a small opening 29a adapted to the size of laboratory animals such that animals may be placed into the container or removed from the container through the opening 29a while the cover 30 is in place. The opening 29a provides a means for moving animals to and from the cage 27 without having to remove the entire cover 30 from the container. In this way, the release of gas from the cage 27 is reduced as animals are moved to and from the container. The opening 29a is adapted to receive a lid 31 which is complementary to the opening. Preferably, the lid 31 conforms to the perimeter of the opening 29a in sealing engagement to minimize the entry or release of gases to or from the container. The anesthetic host cage 27 may further include one or more partitions to subdivide the cage into a plurality of smaller compartments.

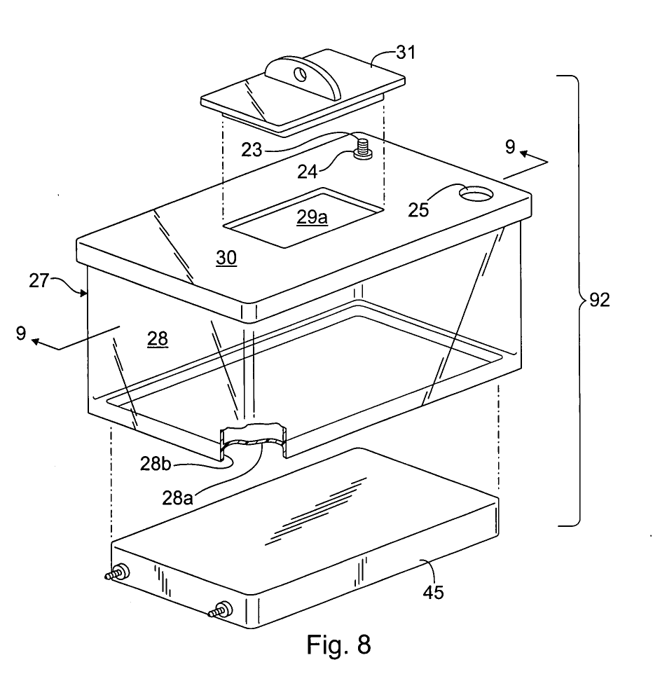

Anesthetic gas mix is delivered to anesthetic host cage 27 via an inlet 24. Referring to FIG. 8, an inlet 24 passes through the cover 30 of the host cage 27. The inlet 24 may alternatively pass through the sidewall 28 of the host cage. The inlet 24 is configured to cooperate with a quick-connect fitting 23. The quick-connect fitting allows laboratory personnel to readily attach and detach alternative conduits that deliver anesthetic gas, pure oxygen, or a combination of the two components to the anesthesia host cage 27.

The host cage 27 is configured to discharge exhaled gas from the interior of the cage as new gas is introduced into the cage. Removal of exhaust gases from the host cage 27 and the surrounding work area protects laboratory personnel from the adverse effects of inhaling anesthetic gas components. Referring to FIG. 8, the host cage 27 is shown with an outlet 25 configured to receive and remove exhaust gas from the interior of the cage. Exhaust gas may be collected form the outlet 25 using a variety of exhaust systems or components. For example, an air filter 29 may be mounted on the outlet 25, as shown in FIGS. 1 and 3. Alternatively, the outlet 25 may release exhaust gas to the exterior of the host cage 27 where it is captured by a fume hood or other exhaust removal system in the laboratory facility. The outlet 25 may also be connected directly to the facility's exhaust system via a conduit to facilitate removal of exhaust gases. Where conduit is used to connect the outlet 28 directly to the facility exhaust system, it may not be desirable or necessary to include a filter on the outlet, unless it is desired to monitor the content of contaminants in the gas discharged from the container. In such case, a filter having a sensor may be used, as described hereinafter.

Referring to FIGS. 10-14, the exhaust system 60 on the group apparatus 92 preferably includes a horizontal negative pressure recapture system to remove exhaust gases from the host cage 27. The exhaust system 60 comprises a plurality of stainless steel trays 61 that are covered by removable perforated steel grids 63. The grid is spaced above the bottom of the tray to provide a compartment which forms a plenum. The grids 63 are configured to support one or more of the solo apparatuses 90 and the group apparatuses 92 during use. The trays 61 are connected to an exhaust manifold 67 by exhaust conduits 65. The exhaust conduits 65 and manifold 67 are further connected to an exhaust fan or other mechanism within the facility's ventilation system that is operable to generate a negative pressure or vacuum in the plenum, the conduits and the manifold. As such, the trays 61 are configured to withdraw exhaust gases from the apparatuses 90, 92 when a negative pressure or vacuum is supplied to the conduits and plenum. Exhaust gases are withdrawn through the grids 63 and conduits where they are discharged from the manifold 67. Manifold 67 may comprise one or more filters, such as HEPA filters, to cleanse the air prior to its release into the general environment.

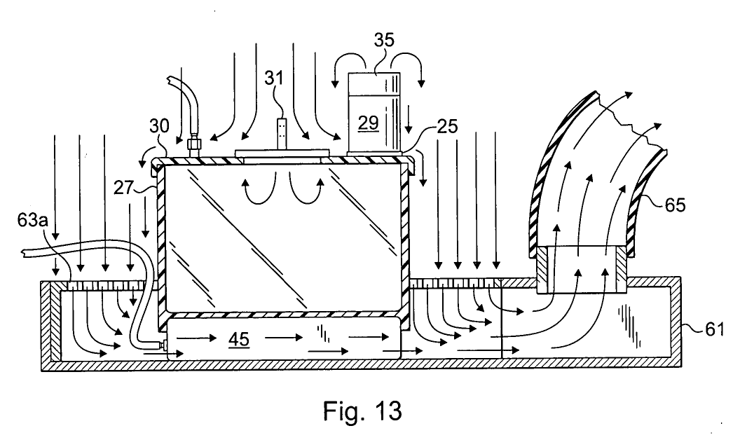

When anesthetic gas is delivered to a group apparatus 92, an alternate grid structure 63a may be used with the exhaust system 60, as shown best in FIGS. 10-12. Grid 63a comprises a centrally located cut out 71 adapted to receive an anesthetic host cage 27. In particular, the perimeter of the cut out 71 is slightly larger than the exterior perimeter of the anesthetic host cage 27 such that the host cage may be partially inserted into the cut out and extend into the tray 61, as shown in FIG. 12. The support base 45 for the host cage 27 may be placed in the tray 61 beneath the cut out 71 to support the host cage, as shown in FIG. 13. By positioning the host cage 27 through the grid 63a in this manner, the required overhead space for the group apparatus 92 is reduced, and the removal of exhaust gases from the host cage 27 is facilitated, as indicated by the plurality of arrows in FIG. 13. Exhaust gases in the host cage 27 exit the cage through the outlet 25 and outlet filter 29. Vacuum pressure supplied through the exhaust conduit 65 creates negative pressure in the tray 61. The negative pressure in the tray 61 causes the exhaust gases to wash past the sides of the container 27, through the grid 63a and into the tray. The exhaust gases are then drawn up through the exhaust conduit 65, as indicated by the arrows in FIG. 13.

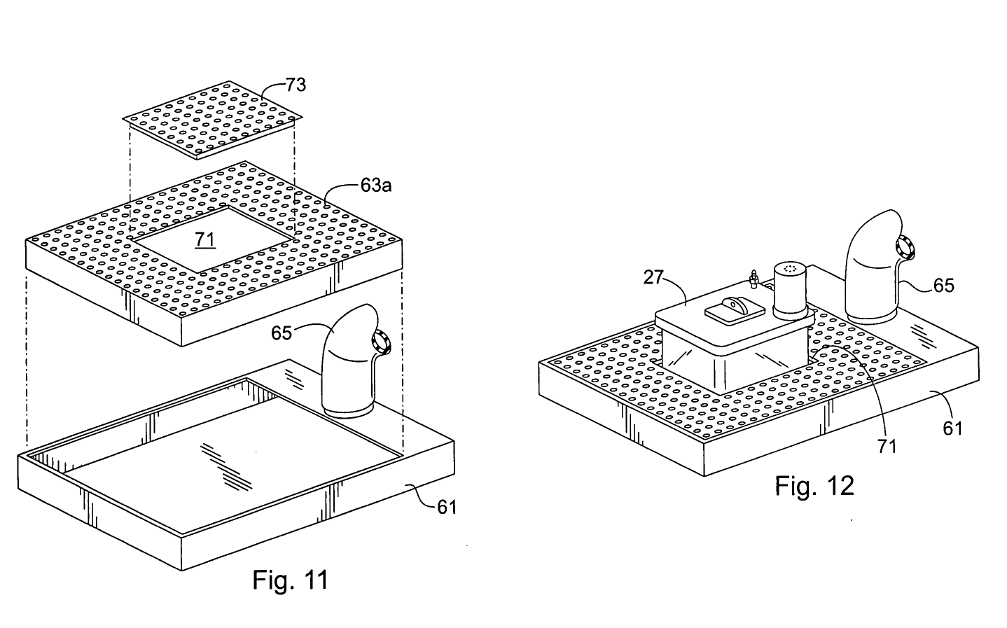

Referring to FIG. 11, a cover plate 73 may be provided for placement over the cut out 71 when a the cut out is not in use. The cut out 71 is configured to receive and support the cover plate 73 in a position such that the top surface of the cover plate is generally flush with the surface of the grid 63a to provide a uniform and substantially uninterrupted grid surface.

The present system 1 may have a thermoregulatory system to control the body temperature of laboratory animals being anesthetized. The thermoregulatory system may be used in connection with one or more solo apparatuses 90 and/or a group apparatuses 92 at one time. Where a thermoregulatory system is used, the support bases 43 and 45 are comprised of hollow containers, as best shown in FIG. 7. The hollow bases 43 and 45 are formed of a heat conductive material. A heat-exchange medium in the bases 43 and 45 may be used to transfer heat by radiation or conduction through the walls of the base to the animal or animals placed on the base. A variety of heat-exchange media may be used in accordance with the present invention. It may be desirable to use water as the heat-exchange medium based on the low cost and general availability of water in laboratory facilities.

Preferably, the bases 43 and 45 are heated by circulating a fluid heat-exchange medium, such as water, through the bases to provide a substantially constant base temperature. Referring to FIGS. 5-7, the bases may comprise an inlet 98 adapted to receive heated water and an outlet 99 configured to discharge heated water from the hollow base. In the base 43 shown in FIG. 7, a longitudinal baffle 43c is provided along the longitudinal centerline of the base to assure longitudinal flow of heat exchange medium through the length of the base 43. The direction of flow of heater water is represented by the arrows 43d, 43e and 43f in FIGS. 5-7. The system 1 may utilize a variety of hydraulic elements and piping configurations, including flow-through systems and closed-loop pressurized systems. Referring to FIG. 1, a solo apparatus 90 and group apparatus 92 are shown connected in series in a closed loop heating system, but the apparatuses may alternatively be connected in parallel in a closed loop system. Where there is an ample supply of hot water, the apparatuses may be connected in an open system in which the hot water may flow through the apparatus and be discharged to waste. In the closed loop system shown in the drawings, a pump 47 is connected to the bases through a network of conduits to recirculate heated water through the bases. The pump 47 may comprise an internal reservoir having a heating element (not shown) connected to an electrical power supply through a power cord 47c. The electrical heating element may be submerged in the reservoir of the pump, and be energized to heat to the water under the control of a thermostat 47a as it flows through the pump 47. The pump 47 discharges the heated water to an influent line 42 where it begins circulating through the system apparatuses. In particular, the influent line 42 connects to an inlet port 44 on support base 45 to discharge heated water into the base. A first discharge line 48 connects to an outlet port 46 on the base 45 and is configured to receive water as it is cycled through the base. Line 48 connects to an inlet port 49 on support base 43 on the solo apparatus 90. As such, line 48 is configured to transfer heated water from base 45 to base 43. A second discharge line 59 connects to an outlet port 51 on the base 43 and returns the water back to the pump 47 where the water is reheated and recirculated. The thermoregulatory system is configured to maintain a desired body temperature of laboratory animals undergoing procedures on the bases 43 and 45 to ensure the viability of the animal and facilitate the successful completion of the procedure. Preferably, the pump 47 contains an adjustable thermostat 47a configured to control the temperature of the water in the thermoregulatory system in accordance with the physical requirements of the particular species of laboratory animal being treated. For example, the thermostat 47a may be operable to shut off the internal heating element when the temperature of water entering the pump reaches a particular temperature. A shut off switch 47b is provided to cut off power to the pump.

In some applications, it may be necessary to discontinue anesthetic gas mix and deliver pure oxygen to the laboratory animal. Pure oxygen is used, for example, to facilitate rapid recovery of an animal after anesthetic gas has been applied. Referring to FIG. 2, an emergency bypass is provided in the system 1 to deliver pure oxygen directly to one or more apparatuses in the system. The oxygen supply 2 includes an emergency bypass junction that connects to a bypass manifold 4. The bypass manifold 4 has an outlet connected to a conduit 41 which connects to one or more apparatuses of the present system. In FIG. 2, the conduit 41 provides an emergency bypass line to a group apparatus 92. Preferably, the conduit 41 has a quick-connect fitting at its remote end which cooperates with compatible fittings on the apparatuses of the system 1 so that the bypass conduit can be readily connected and disconnected to different apparatuses as needed.

Referring to FIG. 3, an attachment 35 may be provided at the discharge end of the filters 17 and 29 to monitor the condition of the filters. More specifically, an attachment 35 may be connected to filters 17 and 29 to measure the concentration of anesthetic component or other contaminants being released through the filter. When the concentration of discharged contaminants exceeds a pre-established safety limit, such as a limit based on federal regulations or OSHA standards, the sensor may send an electronic signal to trigger a visual or an audible or a combined visual and auditory signal indicating that a filter is not operating adequately and should be serviced, or the anesthetic flow regulator requires attention. Preferably, the absorbent material in filters 17 and 29 are packaged in replaceable cartridges, which are easily loaded into and unloaded from filter canisters. In this way, a spent filter cartridge or improperly working filter cartridge may be easily removed and replaced with a new filter.

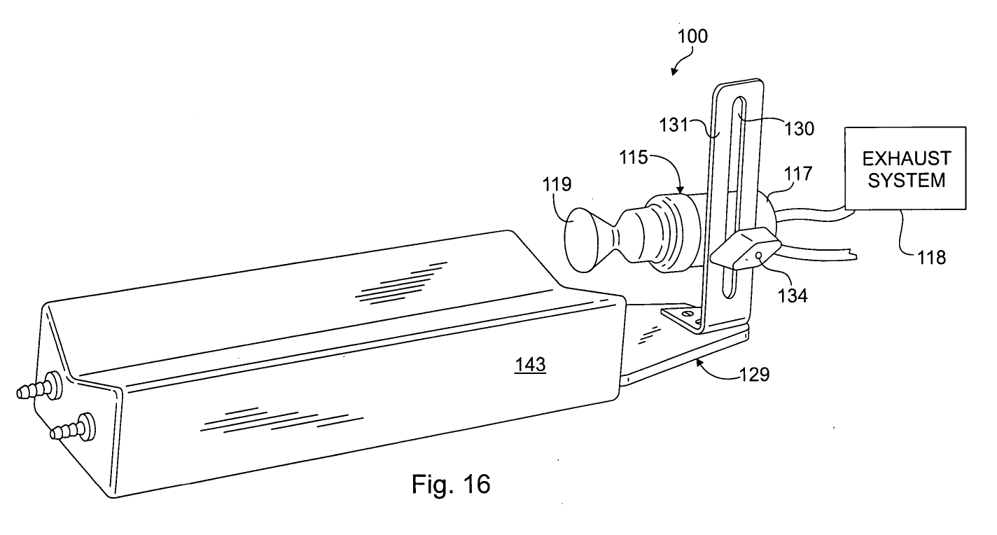

Referring now to FIG. 16, the present invention further provides a portable anesthetizing system 100 for anesthetizing laboratory animals. The portable anesthetizing system 100 comprises elements that are similar to the solo apparatuses 90 discussed earlier and may replace one of the solo apparatuses of FIG. 2. The portable system 100 comprises a breathing device 1 15 connected to a support base 143. Like the support base 43 in the solo apparatus 90, the support base 143 in the portable system may have a number of geometric configurations and connect to a thermoregulatory system for controlling the body temperature of an animal placed on the base. The breathing device 115 comprises a non-rebreathing gas delivery mask 119 attached to a breather chamber or shroud 117. The shroud 1 17 preferably has outer dimensions which are the same as as the outside dimensions of the connector 16a of the chamber 16, so that the shroud may accept any one of the masks 19, 119 and 219. The breather shroud 117 is linked, in turn, to an exhaust system 118. Breather shroud 117 may discharge through an absorbent filter media in the shroud to remove contaminants from the exhausted gas. Preferably, the portable system 100 includes a quick-connect fitting that may be readily connected with a manifold or other component.

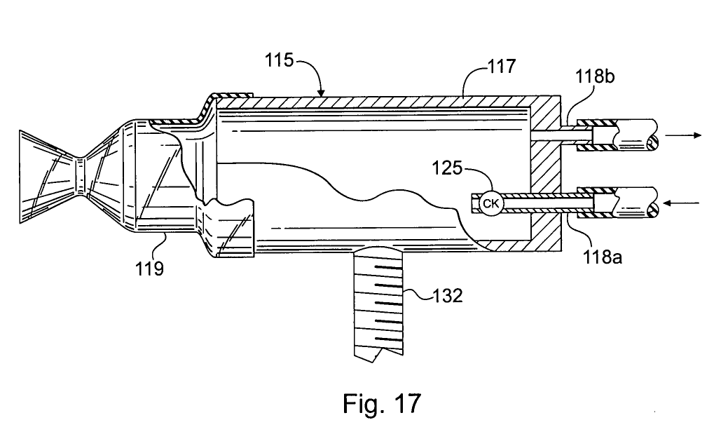

Referring now to FIG. 17, internal elements of the portable breathing device 115 are illustrated. The shroud 117 comprises an inlet hub forming an inlet port 118a through which anesthetic gas is delivered. The shroud also comprises an outlet port 118b through which gas is discharged. A check valve 125 is connected in line with the inlet port 118a to limit the flow of anesthetic gas into the shroud 117. In particular, the check valve 125 is operable in an open position when the laboratory animal inhales and creates a vacuum pressure in the shroud 117. When the animal stops inhalation, the check valve 125 closes to limit further entry and accumulation of anesthetic gas in the shroud 117. Excess gas in the shroud, and any gas exhausted from the animal's nose into the shroud, are discharged through the outlet port 118b. The outlet port 118b may be connected to the exhaust system 60 described earlier, which may or may not include filter canisters and filter sensors. In addition, the shroud 117 may be connected to an emergency bypass system as described earlier to supply pure oxygen to the animal. A ventilator bulb similar to the ventilator 21 shown in FIGS. 1 and 3 may be provided to manually ventilate the portable breathing device 115 during resuscitation of a laboratory animal.

Portable breathing device 115 may be mounted on a support structure 129, as shown in FIG. 16. The support structure 129 provides means to position the gas delivery mask 119 relative to the position of the nose of an animal placed on the portable support base 143. The support structure 129 is comprised of a stand 131 which includes a generally vertical slot 130 and an adjusting nut 134 which cooperates with a threaded stud 132 projecting from the shroud 117 and inserted through the slot. As such, the stud 132 cooperates with the nut 134 to hold the shroud and stand together in frictional engagement. The adjusting nut 134 may be tightened on the stud 132 to fix the delivery mask 119 in a position above the the base 143 to accommodates the particular species of laboratory animal undergoing treatment. The adjusting nut 134 may be loosened on the stud 132 so that the stud is free to slide along the vertical slot 130 in the stand. By sliding the stud in the slot, the breathing device 115 is vertically displaceable to permit adjustment of the mask 119 relative to the animal support base 143. Frictional engagement between the nut 134 and the side of the stand is sufficient to hold the mask in the desired position.

Thus far, the ventilator bulb has been described for use in manual ventilation of the breathing device. The bulbs may also be used as small group induction chamber to administer anesthetic gas to a plurality of small animals placed in the bulb. This arrangement may be desirable when a large group apparatus 92 is not available, or where work space is very limited.

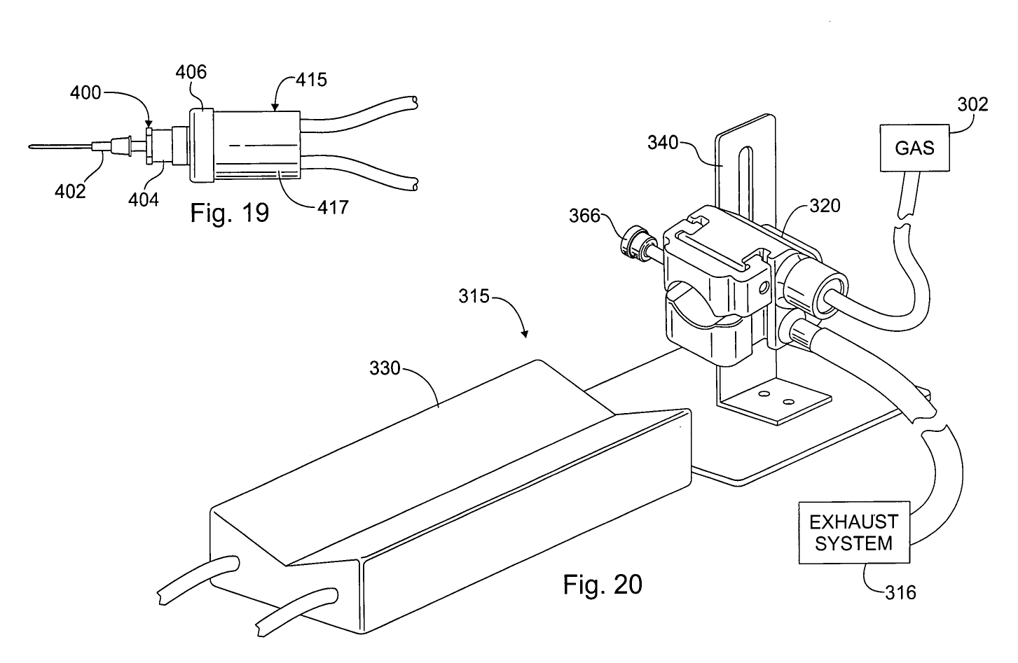

The present invention may be used with intubating tubes to administer anesthetic gas directly to the lungs of an animal during open surgery or other procedures. The components of the present invention preferably include connectors that are cooperable with both breather masks and intubating tubes. This arrangement permits breather masks to be readily substituted with intubating tubes, and vice versa. Referring to FIG. 19, a portable breathing device 415 in accordance with the present invention is connected with an intubating tube assembly 400. The breathing device 415 is similar to the portable breathing device described previously, and includes a generally cylindrical shroud or breather chamber 417. The shroud 417 is connected with an intubating tube assembly 400 that includes an intubating tube 402. The intubating tube 402 connects with a tube base 404 and a large diameter fitting 406. The fitting 406 is configured to connect the tube assembly 400 to the shroud 417. The inner diameter of the fitting 406 is slightly greater than the outer diameter of the shroud, allowing the fitting 406 to connect with the shroud 417 by frictional engagement. The dimensions of the fitting 406 are preferably identical to the dimensions of the fittings associated with the cone-shaped breather masks previously described. In this arrangement, the cone-shaped breather masks and intubating tube are interchangeable and compatible with the same components.

The non-rebreathing feature of the present invention may be incorporated into any of the apparatuses described herein, including but not limited to the solo apparatus and the portable breathing device described above, and the stereotaxic breathing device hereinafter described. The non-rebreathing feature comprises a vacuum-sensitive check valve that permits the delivery of anesthetic gas to the animal only when the animal inhales. This feature of the invention, called “on-demand” gas flow, provides safe and efficient administration of anesthetic gas that prevents harm to the animal and minimizes uncontrolled releases of gas into the surrounding work space.

The check valves in the present invention are sensitive to pressure differentials between the delivery conduits and the interior of the chambers. A variety of pressure-sensitive valves may be used. Each check valve is formed of a flexible material that surrounds a small slit or orifice. The valve is operable in a closed condition, in which the orifice is sealed to prevent gas from flowing through the valve, and an open condition, in which the orifice is unsealed to permit gas to flow through the valve. Preferably, the valve is formed of a resilient flexible material that biases the valve in the closed condition. The valve material is configured to overcome the bias force and open the orifice when the pressure in the inlet port relative to the chamber exceeds a threshold differential. More specifically, the valve material is configured to open the orifice when the animal inhales and creates a vacuum pressure in the breather chamber, while the inlet port behind the valve is under pressure. When the animal stops inhaling, the pressure differential between the inlet port and chamber drops below the threshold differential, and the resilience of the material urges the valve back to the closed position. In this arrangement, the check valve provides a self-regulating feature that administers anesthetic gas to the animal only when the animal inhales.

It has been found that smaller shrouds or air exchange chambers provide rapid delivery of anesthetic gas precisely when the animal inhales. For portable breathing apparatuses, the volume of the shroud is preferably between 1.0-4.0 cubic centimeters. The small volume in the shroud allows the pressure in the shroud to drop rapidly when the animal inhales. The rapid drop in pressure triggers the check valve to open virtually instantaneously as the animal inhales. As a result, the check valve is more responsive to the animal's respiratory rate when the volume of the chamber is relatively small.

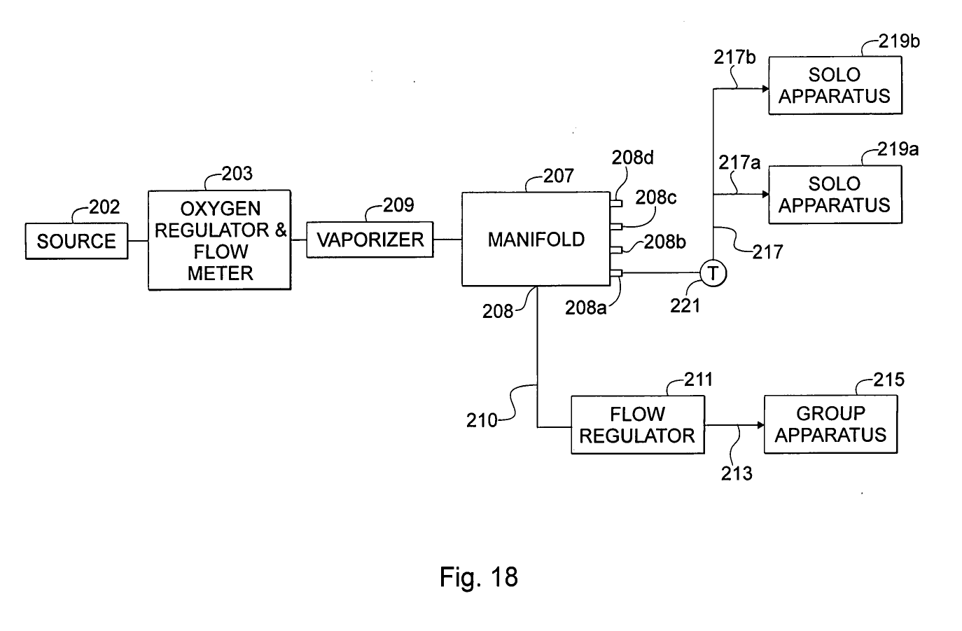

The “on-demand” gas flow feature permits gas to be administered uniformly to multiple apparatuses and breathing devices, without significant pressure variations caused by head losses associated with different conduit lengths and other variables. This advantage can be better understood in conjunction with FIG. 18, which illustrates a system 201 that delivers anesthetic gas mixture to a group apparatus 215 and two solo apparatuses. Anesthesia system 201 includes a source 202 of carrier gas which is connected with an oxygen regulator 203. The source 202 is configured to deliver a carrier gas under pressure into the regulator 203. The regulator 203 is connected to a vaporizer 209 which introduces anesthetizing components to the stream of carrier gas. The vaporizer 209 is connected to a manifold 207, which is adapted to receive the anesthetic gas mixture from the vaporizer. The manifold 207 has a single port 208 which is adapted to be connected to a large conduit 210. The large conduit 210 connects with a flow regulator 211 that incorporates a throttling mechanism. The flow regulator 211 connects with an output conduit 213 which, in turn, connects with a group apparatus 215. The manifold 207 additionally has multiple ports 208a through 208d, each of which is adapted to be connected to other apparatuses. Conduit 208a is connected with a small conduit 217, which splits into two separate lines 217a, and 217b. Line 217a is connected with a solo apparatus 219a, and line 217b is connected with a solo apparatus 219b.

In a typical installation, the supply of gas to the manifold 207 is at a pressure of 10-12 mm Hg. Most of this pressure is initially distributed to the group apparatus 215 when the flow regulator 211 is fully opened to anesthetize a plurality of animals in the group apparatus. Once the animals in the group apparatus 215 are anesthetized, the flow regulator 211 is throttled down to deliver a low flow of gas to the group apparatus sufficient to maintain the animals in an anesthetized state. At this stage, most of the pressure is cut off from the group apparatus, and is distributed back to the manifold where it is redistributed to line 217 and the solo apparatuses 219a, 219b. After animals are placed in the solo apparatuses 219a, 219b, the check valves in the solo apparatuses intermittently open and close in response to each animal's breathing pattern. The intermittent closing of the check valves allows additional back pressure to develop in lines 217a and 217b, eventually reaching a pressure equilibrium. In this arrangement, the available back pressure for each solo apparatus 219a and 219b is the same. This is the case even where line 217b has a much greater length than line 217a. As a result, the gas flow in each line is not affected by head losses associated with the length of the line, permitting each animal to receive the same flow of gas when the animal inhales. The system is configured to supply anesthetic gas mixture at uniform pressures to a plurality of solo apparatuses connected in parallel with the line 217, creating a level plane of anesthesia that can be administered without the need for pressure regulators at each solo apparatus.

The check valves may not operate properly if they are subject to significant back pressures in lines 217a, 217b. To ensure proper operation of the check valves, the port 208a and the conduit 217 have smaller orifices and diameters than port 208 and line 210. The smaller orifices and diameters in the port 208a and conduit 217 throttle the flow and reduce the pressure of the gas mixture supplied to the solo apparatuses. In the preferred embodiment, the port 208a and conduit 217 are configured to throttle the flow to less then one-fifth of the pressure in the manifold. For most applications, the desired pressure supplied to the solo apparatuses is preferably less than 1 mm Hg. An optional throttle valve 221 may be interposed in the conduit 217 to lower the pressure in lines 217a and 217b to the desired pressure.

Referring now to FIGS. 20-25, an alternate breathing apparatus 315 is shown in accordance with the present invention for use in stereotaxic and non-stereotaxic procedures. The breathing apparatus 315 includes an anesthesia mask or enclosure 320 connected to a supply of anesthetic gas 302. The enclosure 320 is adapted to fit over the nose of an animal to deliver anesthetic gas to the animal during a stereotaxic or non-stereotaxic procedure. A heated bed 330 supports the animal during the procedure. The enclosure 320 is configured for use in two modes: (1) as part of a stereotaxic unit, or (2) as a stand-alone breathing device. In FIG. 20, the enclosure 320 is supported by a stand 340 for use as a stand-alone breather unit. The stand 340 supports the enclosure 320 and provides a means for adjusting the position of the mask so that it aligns with the animal's nose when the animal is positioned on the bed 330.

Referring now to FIGS. 20-22, the enclosure 320 has a generally hollow interior and includes an inlet port 322 and an exhaust port 324 that extend through one side of the mask to connect with the hollow interior. The inlet port 322 connects with the supply of anesthetic gas 302 and permits anesthetic gas to enter the interior of the enclosure 320. The outlet port 324 connects with an exhaust system 316, such as a filter canister or other means for safely exhausting gas from the enclosure 320. The enclosure 320 may be used with a variety of components operable to deliver and remove gas from the interior of the enclosure.

The enclosure 320 includes a hollow base portion 350 and a head restraint portion 360 that connects with the base portion. The base 350 connects with the head restraint 360 to form a nose aperture or receptacle 370 on one side of the mask. The nose aperture 370 is configured to extend around an animal's nose and provide a means for delivering anesthetic gas to the animal. The base 350 and head restraint 360 may be formed in a variety of shapes to form the nose aperture 370. For example, the base 350 may include a first saddle portion 352, and the head restraint 360 may include a second complementary saddle portion 362 that aligns with the first saddle portion 352 to form an elliptical nose aperture.

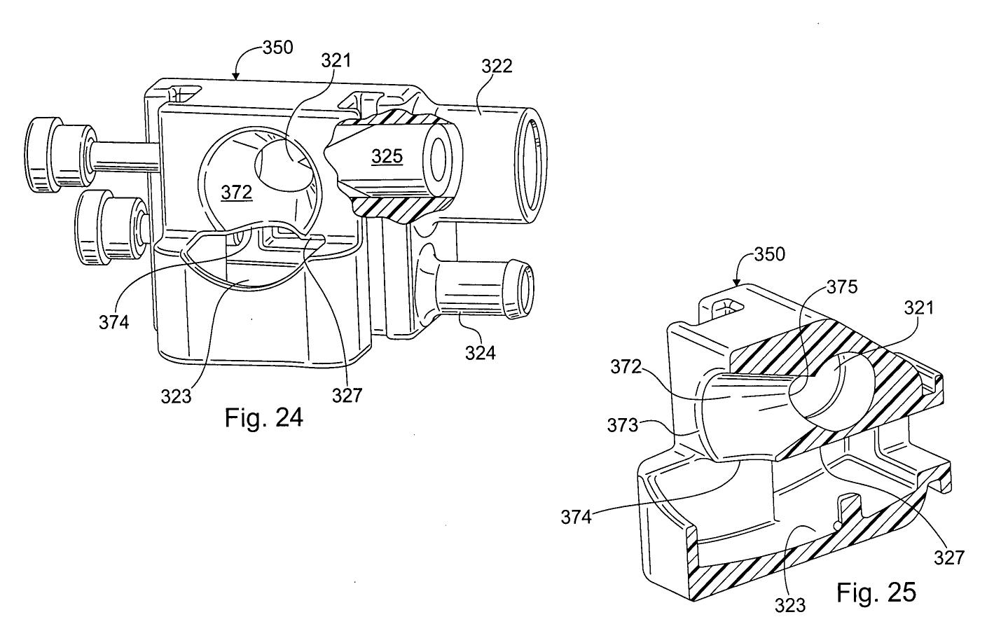

The breathing apparatus 315 is a non-rebreathing apparatus, similar to solo apparatuses and breathers described above. That is, the enclosure 320 is configured to deliver gas through an inner chamber to the animal's nose, while substantially preventing the flow of exhaled gases from the animal's mouth back into the same chamber. A number of structural configurations may be used to divert exhaled gases away from the inner chamber that delivers gas to the animal's nose. Referring to FIGS. 24-25, a barrier 327 extends through the interior of the base 350 to form an upper inhalation chamber 321 and a lower exhaust chamber 323. A cone-shaped or funnel-shaped conduit 372 extends through the base 350 and connects with the upper inhalation chamber 321. The cone-shaped conduit 372 forms a fluid path between the nose aperture 370 and the upper inhalation chamber 321 when the head restraint 360 is connected with the base 350. The conduit 372 has a wide conduit end 373 that is positioned in proximity to the nose aperture 370, and a narrow conduit end 375 that connects with the inhalation chamber 321. The distance between the wide end 373 and the narrow end 375 of the conduit 372 is preferably less than the length of the animal's facial structure so as to permit the anterior portion of the animal's nose to project out of the narrow end and into the inhalation chamber 321. The animal's nose need not reach or project into the inhalation chamber 321 to receive anesthetic gas, however.

The conduit 372 includes a notch 374 formed through a bottom section of the conduit. The notch 374 is configured to align with the animal's mouth when the animal's nose is inserted into the nose aperture 370. The notch 374 provides a fluid path between the animal's mouth and the lower exhaust chamber 323. In this arrangement, the animal inhales gas from the inhalation chamber 321 through the nose and exhales gas out of the mouth into the lower exhaust chamber 323. Backflow of exhaled gases into the inhalation chamber 321 is substantially limited, since the distance between the animal's mouth and the narrow end of the conduit 372 is significantly greater than the distance between the animal's mouth and the notch 374. Moreover, exhaled gases in the exhaust chamber are substantially prevented from entering the upper inhalation chamber by the barrier 327.

Referring now to FIG. 24, the inlet port 322 extends from the upper portion of the base 350 in alignment with the inhalation chamber 321, and the outlet port 324 extends from the lower portion of the base in alignment with the exhaust chamber 323. In this arrangement, the inlet port 322 introduces gas into the inhalation chamber 321, and the outlet port removes gas from the exhaust chamber 323.

The enclosure 320 has an on-demand feature that permits the flow of anesthesia gas into the inhalation chamber 321 only when the animal inhales. The inlet port 322 on the base 350 connects with a vacuum-sensitive check valve 325 that controls the delivery of gas into the inhalation chamber 321. The vacuum-sensitive check valve 325, which is shown schematically in FIG. 24, extends from the inlet port 322 into the inhalation chamber 321 and is operable in response to pressure changes in the inhalation chamber 321. More specifically, the on-demand valve 325 is operable between an open position to facilitate inflow of anesthetic gas into the inhalation chamber 321, and a closed position to prevent anesthetic gas from entering the inhalation chamber. The on-demand valve 325 is normally biased in the closed position in the absence of vacuum pressure in the inhalation chamber 321. When the animal inhales to create a vacuum pressure in the inhalation chamber 321, the on-demand valve 325 adjusts to the open position to permit the entry of anesthetic gas into the inhalation chamber. The on-demand valve 325 may be any type of one-way valve or check valve that is sensitive to pressures in the breathing chamber.

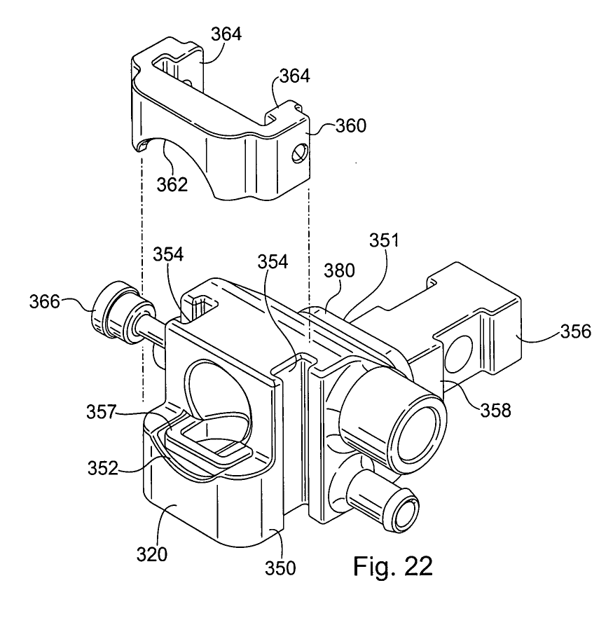

Referring now to FIG. 22, the base 350 has a pair of grooves 354 extending along the exterior of the base. The head restraint 360 has a pair of corresponding flanges 364 that conform with the shape of the grooves 354 on the base 350. The grooves 354 on the base 350 are configured to provide a guide for connecting the head restraint 360 with the base. More specifically, the grooves 354 on the base 350 are adapted to receive the flanges 364 on the head restraint 360 in a sliding engagement that permits the head restraint to slide onto the base. In this arrangement, the animal's nose can be placed over the first saddle portion 352 of the base 350, and the head restraint 360 can be slid down over the animal's head to secure the animal's head in the aperture 370. The head restraint 360 and cone-shaped conduit 372 cooperate to form a brace that encloses the animal's head and limits the animal's ability to move its head. Preferably, the head restraint 360 includes a restraint lock screw 366 which extends through the restraint and frictionally engages the base 350 when the restraint is connected with the base. The lock screw 366 can be tightened to secure the head restraint 360 on the base 350, and loosened to permit the head restraint to be slidably adjusted or removed from the base.

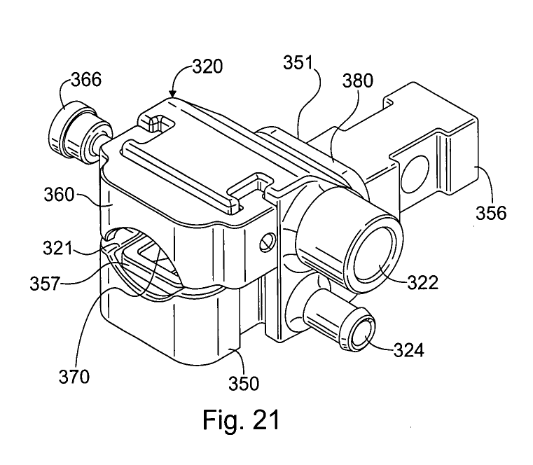

The enclosure 320 is configured for use with a variety of stereotaxic accessories. Referring to FIGS. 21-23, a tooth bar 356 is inserted into the lower exhaust chamber 323 of the enclosure 320. The tooth bar 356 comprises a bar extension 357 and an elongate body 358 that can be inserted into the enclosure 320 through a rear opening 351 that is aligned with the lower exhaust chamber. When the tooth bar 356 is inserted into the enclosure 320, the bar extension 357 extends generally horizontally across the first saddle portion 352 of the base 350. In this position, the tooth bar 356 extends in proximity to the animal's mouth. The animal's front teeth are secured over the bar to limit movement of the animal's head and insure that the animal's nose remains in the inhalation chamber 321.

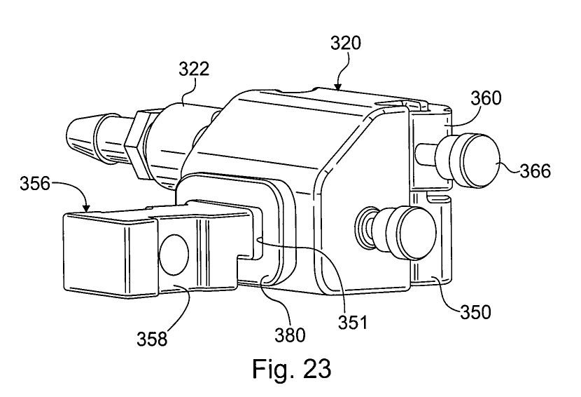

The breathing apparatus 315 is configured to prevent the uncontrolled release of anesthesia gas into the atmosphere. As stated earlier, the device includes an on-demand feature that draws gas from the inlet port only when the animal inhales. Since gas flow over the animal's nose is not a constant flow, little or no anesthetic gas mixture is permitted to bypass the animal's nose when the animal is not inhaling. In addition, the enclosure 320 is sealed to substantially prevent the uncontrolled release of anesthesia gas into the atmosphere. Two different seals are provided to seal the rear opening 351 of the enclosure. When the tooth bar 356 is inserted into the mask for stereotaxic procedures, a gasket seal 380 is placed in the rear opening, as seen best in FIG. 23. The gasket seal 380 attaches to the perimeter of the rear opening 351 and has a central opening adapted to receive the tooth bar 356. The gasket seal 380 seals the space between the walls of the rear opening and the tooth bar 356, substantially preventing the release of gas from the chamber 321. When the tooth bar 356 is not used with the enclosure, the rear opening is sealed with a plastic cover (not shown) that seals the entire rear opening.

The enclosure 320 may formed of a variety of materials. Preferably, the base 350, head restraint 360, and related components are formed of one or more radiolucent materials that permit passage of radiation or x-rays. In this way, radiographic images of the animal can be obtained while the apparatus 315 is in use, with minimal obstruction or visual interference from the enclosure and associated components. For example, an enclosure 320 formed of radiolucent material may be used with horizontal or vertical magnetic resonance imaging (MRI) devices. A variety of radiolucent materials may be used, including but not limited to composites and thermoplastics.

The operation of the breathing apparatus 315 will now be described as it is used in a typical stereotaxic procedure. The animal is placed on a bed or other support means, with the animal's head aligned with the enclosure 320. The head restraint 360 is removed from the base 350, and the animal's head is placed in the cone-shaped conduit 372 with the tip of the animal's nose projecting through the narrow end of the conduit into the inhalation chamber 321 (hereinafter, the “breathing position”). Using the tooth bar 356, ear bars, or other stereotaxic components known in the art, the animal's head is immobilized in the breathing position. The flanges of the head restraint 360 are inserted into the grooves on the base 350, and the restraint is slid down over the animal's head to enclose the animal's head. The restraint lock screw 366 on the head restraint is tightened to secure the head restraint 360 on the base 350. The inlet port 322 is connected with a supply of anesthetic gas, and the outlet port is connected with an exhaust system 316.

The anesthetic gas supply is activated to deliver pressurized gas to the enclosure 320, where it backs up behind the check valve 325 at the inlet port 322. When the animal inhales, negative pressure is created in the inhalation chamber 321. When the pressure differential between the pressure in the chamber and behind the check valve reaches the threshold differential, the on-demand valve 325 moves to the open position. In the open position, anesthetic gas enters the inhalation chamber 321, where it is inhaled by the animal. When the animal stops inhaling and begins exhaling, the check valve 325 closes, preventing further inflow of anesthetic gas into the inhalation chamber 321. In this arrangement, the check valve 325 substantially prevents the inflow of anesthetic gas into the breathing chamber 321 when the animal is exhaling, so that the animal's ability to exhale anesthetic gas and carbon dioxide is not inhibited. Exhaled gas is conveyed through the notch 374 into the exhaust chamber 323 and exits the apparatus via the outlet port to the exhaust system 316.

The terms and expressions which have been employed are used as terms of description and not of limitation. There is no intention in the use of such terms and expressions of excluding any equivalents of the features shown and described or portions thereof. It is recognized, therefore, that various modifications are possible within the scope and spirit of the invention. Accordingly, the invention incorporates variations that fall within the scope of the following claims.

We claim:

1. An anesthesia system for administering an anesthetic gas to the nose of an animal, said anesthesia system comprising:
A. a mask having a receptacle adapted to surround and form a fluid path to the animal's nose;
B. a breather enclosure forming an air exchange chamber, said breather enclosure comprising a connector configured for coupling with said mask to connect the receptacle and air exchange chamber in fluid communication;
C. an inlet hub extending inside said air exchange chamber and forming an inlet port through a wall in the breather enclosure, said inlet port being adapted to convey the anesthetic gas into the chamber and to the mask; and
D. a check valve connected with the inlet hub and extending within said air exchange chamber, said check valve being operable in response to relative pressure in the air exchange chamber between an open position, which permits anesthetic gas to enter the air exchange chamber, and a closed position, which substantially prevents anesthetic gas from entering the air exchange chamber,
wherein, the check valve moves to the open position in response to vacuum pressure created when the animal inhales, said check valve being normally biased in the closed position.

2. The anesthesia system of claim 1 comprising a cone-shaped receptacle on the mask, said cone-shaped receptacle adapted to receive and conform with the contour of the animal's nose to minimize void space between the animal's nose and the mask.

3. The anesthesia system of claim 1, wherein the mask comprises a resilient flexible material adapted to deflect and conform to the contour of the animal's nose.

4. The anesthesia system of claim 1, wherein the breather enclosure comprises a connector and the mask comprises a connecter end configured to detachably couple the mask with the connector on the breather enclosure.

5. The anesthesia system of claim 4, comprising an intubating tube having a tube end configured for detachable coupling with the connector on the breather enclosure to enable substitution of the mask with said intubating tube.

6. The anesthesia system of claim 1, wherein the volume of the air exchange chamber is approximately between 1.0-4.0 cubic centimeters.

7. A self-regulating on-demand breathing apparatus for administering an anesthetic gas to the nose of an animal, said breathing apparatus comprising:
A. an enclosure forming a chamber and comprising an inlet hub extending within said chamber, said inlet hub forming an inlet port through a wall of the enclosure adapted to convey the anesthetic gas through the wall into the chamber;
B. a receptacle extending from the enclosure and forming a fluid path between the animal's nose and the chamber; and
C. a check valve connected with the inlet hub and extending within said chamber adjacent the receptacle, said check valve being operable in response to relative pressure in the chamber between an open position, which permits anesthetic gas to enter the chamber, and a closed position, which substantially prevents anesthetic gas from entering the chamber,
wherein the check valve moves to the open position in response to vacuum pressure created when the animal inhales to administer the anesthetic gas in response to the animal's breathing, said check valve being normally biased in the closed position.

8. The self-regulating breathing apparatus of claim 7, wherein the receptacle comprises a first cone-shaped mask adapted to receive and conform with the contour of the animal's nose to minimize void space between the first animal's nose and the mask.

9. The self-regulating breathing apparatus of claim 8, wherein the enclosure comprises a connector and the first mask has an end configured to detachably couple the first mask with the connector on the enclosure.

10. The self-regulating breathing apparatus of claim 9, comprising an intubating tube having a tube end configured for detachable coupling with the connector on the enclosure to enable substitution of the mask with said intubating tube.

11. The self-regulating breathing apparatus of claim 9, comprising a second mask configured for detachable coupling with the connector on the enclosure to enable substitution of the first mask with the second mask.

12. The self-regulating breathing apparatus of claim 11, wherein the first mask is configured to conform to the anatomy of a first species of animal, and the second mask is configured to conform to the anatomy of a second species of animal.

13. The self-regulating breathing apparatus of claim 7, wherein the receptacle comprises a base portion extending from the enclosure and a head restraint portion detachably coupled with the base portion.

14. The self-regulating breathing apparatus of claim 13 comprising a first saddle portion on the base portion and a second saddle portion on the head restraint portion, said second saddle portion being configured for complementary alignment with said first saddle portion to form an elliptical nose aperture.

15. The self-regulating breathing apparatus of claim 13, wherein the enclosure comprises a centrally located barrier, said enclosure forming an inhalation chamber and an exhaust chamber separated from the inhalation chamber by said barrier, said inlet hub and check valve extending within said inhalation chamber.

16. The self-regulating breathing apparatus of claim 7 comprising a radiolucent material.

17. The self-regulating breathing apparatus of claim 7, wherein the volume of the chamber is approximately between 1.0-4.0 cubic centimeters.

18. A breathing apparatus for administering an anesthetic gas to the nose of an animal, said breathing apparatus comprising:
A. an enclosure having a hollow interior and a barrier extending through the hollow interior to form an inhalation chamber and an exhaust chamber;
B. a first sidewall on the enclosure forming an inlet port that extends through the first sidewall in fluid communication with the inhalation chamber;
C. a second sidewall on the enclosure forming an exhaust port that extends through the second sidewall in fluid communication with the exhaust chamber;
D. a receptacle extending from the enclosure and forming a fluid path between the animal's nose and the inhalation chamber; and
E. a check valve extending from the inlet port and projecting into the inhalation chamber adjacent the receptacle, said check valve being operable in response to vacuum pressure created in said inhalation chamber when the animal inhales,
wherein the check valve is configured to move to an open position in response to vacuum pressure created in the inhalation chamber when the animal inhales to permit anesthetic gas to enter the inhalation chamber, and to a closed position, in which the check valve prevents the entry of anesthetic gas into the inhalation chamber, said check valve being normally biased in the closed position.

19. The breathing apparatus of claim 18, wherein the receptacle comprises a base portion and a head restraint portion configured to connect with the base portion around the animal's head.

20. The breathing apparatus of claim 19, wherein the base comprises a pair of longitudinal grooves and the head restraint comprises a pair of flanges configured to slidably engage said grooves to detachably connect the head restraint with said base.

21. The breathing apparatus of claim 19 comprising a restraint lock screw extending through the head restraint and configured to frictionally engage the base when the head restraint is connected with the base to secure the head restraint in a fixed position relative to the base.

22. The breathing apparatus of claim 19 comprising a first saddle portion on the base portion and a second saddle portion on the head restraint portion, said second saddle portion being configured for complementary alignment with said first saddle portion to form an elliptical nose aperture.

23. The breathing apparatus of claim 19, wherein the enclosure forms a funnel-shaped conduit having a wide conduit end adjacent to the receptacle, and a narrow conduit end connected with the inhalation chamber.

24. The breathing apparatus of claim 23, wherein the narrow conduit end is adapted to receive the anterior end of the animal's nose and permit said anterior end to project out of the conduit into the inhalation chamber.

25. The breathing apparatus of claim 18, wherein the barrier forms a notch to connect the conduit and the exhaust chamber in fluid communication.

26. The breathing apparatus of claim 18, wherein the first sidewall is the same as the second sidewall.

27. An anesthesia system for administering an anesthetic gas to a plurality of animals comprising:
A. a source of a pressurized gas operable to produce a flow of pressurized gas;
B. a vaporizer fluidly connected to said source of pressurized gas and adapted to introduce an anesthetic component into the flow of pressurized gas to create an anesthetic gas mixture;
C. a manifold fluidly connected to said vaporizer to receive said anesthetic gas mixture, said manifold comprising a first outlet port and a second outlet port, said first and second outlet ports being operable to deliver the gas mixture through said ports simultaneously;
D. a first conduit connected with the first outlet port, and a second conduit connected with the second outlet port;
E. a group apparatus connected with the first conduit for administering the anesthetic gas to a first group of animals;
F. a plurality of solo apparatuses connected with said second conduit in a parallel arrangement to deliver the gas mixture to a second group of animals, each of said solo apparatuses comprising a breather adapted for connection with up to one of said second group of animals; and
G. a flow regulator connected in the first conduit between said manifold and said group apparatus, said flow regulator comprising a throttling mechanism operable to decrease the flow of gas into the group apparatus and build a back pressure of gas in the manifold, said second conduit being configured to evenly distribute said back pressure to the solo apparatuses such that each solo apparatus is supplied with an equal gas pressure.
